(19) United States
(12) Patent Application Publication
(10) Pub. No.: US 2026/0048154 A1
(43) Pub. Date: Feb. 19, 2026

(54) CORE-SHELL NANOPARTICLE HAVING A NITROGEN-VACANCY NANODIAMOND CORE SURROUNDED BY AN UPCONVERSION NANOPARTICLE SHELL FOR ENHANCED QUANTUM SENSING AND LASER COOLING

(71) Applicant: Toyota Motor Engineering & Manufacturing North America, Inc., Plano, TX (US)

(72) Inventors: Shahriar Esmaeili, Ann Arbor, MI (US); Paul Schmalenberg, Ann Arbor, MI (US); Ercan Dede, Ann Arbor, MI (US)

(73) Assignees: Toyota Motor Engineering & Manufacturing North America, Inc., Plano, TX (US); Toyota Jidosha Kabushiki Kaisha, Aichi-ken (JP)

(21) Appl. No.: 18/806,185

(22) Filed: Aug. 15, 2024

(51) Int. Cl. A61K 49/18; A61K 49/04; B82Y 5/00

(52) U.S. Cl. CPC A61K 49/183; A61K 49/04; B82Y 5/00

(57) ABSTRACT

A core-shell nanoparticle having a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation is disclosed. Integration of cleaned NVNDs with UNCPs for enhancing optical manipulation and enabling efficient energy transfer for applications in biological imaging, quantum sensing and laser cooling is disclosed.

### TECHNICAL FIELD

The present disclosure generally relates to quantum sensing and cooling applications through combination of nitrogen-vacancy nanodiamonds and upconversion nanoparticles. In particular, a core-shell nanoparticle having a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation.

### BACKGROUND

The background description provided herein is for the purpose of generally presenting the context of the disclosure. Work of the presently named inventors, to the extent it may be described in this background section, as well as aspects of the description that may not otherwise qualify as prior art at the time of filing, are neither expressly nor implicitly admitted as prior art against the present technology.

Quantum sensing techniques utilize principles from quantum mechanics to make highly precise measurements of physical quantities such as magnetic fields, time, and acceleration. These techniques rely on the unique behavior of quantum systems, such as superposition and entanglement, to achieve sensitivity and accuracy beyond what classical sensors can achieve.

Quantum sensors are devices that utilize quantum phenomena to measure physical quantities with precision and sensitivity. These sensors rely on the principles of quantum mechanics, such as superposition, entanglement, and quantum coherence, to detect and measure various physical properties. Quantum sensors have numerous potential applications, including in navigation, mineral exploration, medical imaging, and fundamental physics research.

Quantum cooling refers to the process of cooling a physical system, such as atoms or ions, to extremely low temperatures using quantum mechanical principles. The aim is to bring the system to its quantum ground state, where classical thermal effects are minimal, and quantum effects dominate.

Quantum cooling is applicable in the field of quantum technologies, such as quantum computing, quantum simulation, and quantum metrology. By cooling atoms or ions to their quantum ground state, quantum systems can be manipulated and controlled with high precision, enabling the implementation of quantum algorithms and the study of novel quantum phenomena.

One method of quantum cooling is laser cooling, which involves using laser light to slow down and cool atoms or ions. This technique exploits the momentum exchange between photons and atoms. When an atom absorbs a photon from a laser beam, it gains momentum in the direction of the photon's propagation. By tuning the laser frequency slightly below an atomic transition, atoms moving toward the laser beam preferentially absorb photons, slowing down in the process due to the momentum transfer and resulting in cooling of the atomic ensemble.

Nitrogen-vacancy centers in diamonds have been studied for quantum sensing and quantum cooling applications. Nitrogen vacancies form in diamonds when a nitrogen atom replaces a carbon atom in a diamond lattice, leaving a vacant site nearby. Nitrogen-vacancy centers in diamonds exhibit unique electronic and optical properties. The nitrogen vacancy in diamond has several electronic states, including a ground state and several excited states. However, current quantum-sensing and laser cooling systems based on nitrogen-vacancy centers in diamonds have limitations due to the direct optical excitation inefficiency of the nitrogen-vacancy centers in nanodiamonds and the high cost of diamond-based materials.

It would be desirable to address limitations of current quantum technologies based on nitrogen-vacancy centers in diamonds by providing a more efficient, versatile and cost effective approach, and to significantly improve optical properties and energy transfer efficiencies in nitrogen-vacancy diamond based technologies expanding the application spectrum into advanced quantum sensing and biolabeling.

### SUMMARY

This section provides a general summary of the disclosure and is not a comprehensive disclosure of its full scope or all of its features.

In various aspects, the present teachings provide a core-shell nanoparticle having a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation is disclosed.

In some aspects of the core-shell nanoparticle, the UCNP shell further comprises gadolinium (Gd).

In another aspect of the disclosed core-shell nanoparticle, the cleaned nitrogen-vacancy nanodiamond (NVND) comprises a nitrogen-vacancy (NV) center, and a distance (r) between the nitrogen-vacancy center and the UCNP ranges from about 2 nm to about 10 nm.

In another aspect, a film comprising a core-shell nanoparticle, wherein the core-shell nanoparticle comprises a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation is disclosed. In some aspects of the core-shell nanoparticle, the UCNP shell further comprises gadolinium (Gd).

In another aspect, a suspension comprising a core-shell nanoparticle, wherein the core-shell nanoparticle comprises a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation is disclosed. In some aspects of the core-shell nanoparticle, the UCNP shell further comprises gadolinium (Gd).

In another aspect, a bio-labeled molecule for detection of red-emission comprising a biomolecule labeled with a core-shell nanoparticle, wherein the core-shell nanoparticle comprises a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation is disclosed. In some aspects of the core-shell nanoparticle, the UCNP shell further comprises gadolinium (Gd). In another aspect, the bio-labeled molecule for detection of red-emission can be detected at a biological tissue depth ranging from about 1 millimeter to about 1 centimeter.

In another aspect, a bio-labeled molecule for quantum laser-cooling comprising a biomolecule labeled with a core-shell nanoparticle, wherein the core-shell nanoparticle comprises a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), wherein the core-shell nanoparticle emits a red-emission upon a near-infrared (NIR) excitation and wherein the bio-labeled molecule enables laser cooling ranging from about 5° C. to about 25° C. in physiological media is disclosed. In some aspects of the core-shell nanoparticle, the UCNP shell further comprises gadolinium (Gd).

In another aspect, a quantum-sensing molecule comprising a core-shell nanoparticle, wherein the core-shell nanoparticle comprises a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation is disclosed. In some aspects of the core-shell nanoparticle, the UCNP shell further comprises gadolinium (Gd).

In another aspect, the core-shell nanoparticle is applicable in a biomedical imaging device for detecting and imaging biological tissues, a quantum computing device for quantum information processing, a precision meterology device for high-resolution measurements, a magnetic field sensor for detecting and mapping magnetic fields, a single photon detector for quantum communication and cryptography, a laser cooling system for achieving low temperatures in quantum systems, or an atom interferometry device for precision measurements and fundamental physics experiments.

In these different aspects, the core-shell nanoparticle, with an NVND core and LiYF₄:Yb, Er UCNP shell, is excited by near-infrared (NIR) emission at about 980 nm In these different aspects, the near-infrared (NIR) excitation is about 980 nm when the UNCP comprises ytterbium (Yb). In these different aspects, the near-infrared (NIR) excitation is about 808 nm for a UNCP comprising neodymium (Nd).

In these different aspects, the upconversion nanoparticle (UCNP) upconverts the near-infrared (NIR) excitation of the UCNP shell to a UCNP-emission of a green-emission ranging from about 523 nm to about 555 nm, a blue-emission ranging from about 450 nm to about 475 nm, or a red-emission of about 650 nm.

In these different aspects, the red-emission from the core-shell nanoparticle ranges from about 630 nm to about 645 nm.

In these different aspects, the core-shell nanoparticle comprises a Forster Resonance Energy Transfer (FRET) efficiency ranging from about 30% to about 70%. In these different aspects, a diameter of the core-shell nanoparticle ranges from about 10 nm to about 200 nm.

In some aspects, the UCNP comprises a ratio of Y:Yb:Er ranging from about 70:25:5 to about 78:20:2 by weight.

Further areas of applicability and various methods of enhancing the above technology will become apparent from the description provided herein. The description and specific examples in this summary are intended for purposes of illustration only and are not intended to limit the scope of the present disclosure.

### BRIEF DESCRIPTION OF DRAWINGS

The present teachings will become more fully understood from the detailed description and the accompanying drawings wherein:

FIG. 1 is a schematic cross-sectional view of an example of a near-infrared (NIR) excited core-shell nanoparticle of the present disclosure having a cleaned nanodiamond (ND) core and a LiYF₄:Yb, Er UNCP shell, demonstrating the energy transfer process leading to red-emission from nitrogen-vacancy (NV) centers.

FIG. 2 is a schematic view of a proposed application of disclosed core-shell nanoparticle film in a device illustrating a device substrate (Si, SiC, etc.), seed layer (Ti/Au), templated nanowires (eg. TiO₂) and film of deposited core-shell nanoparticles.

It should be noted that the figures set forth herein are intended to exemplify the general characteristics of the methods, algorithms, and devices among those of the present technology, for the purpose of the description of certain aspects. These figures may not precisely reflect the characteristics of any given aspect and are not necessarily intended to define or limit specific examples within the scope of this technology. Further, certain aspects may incorporate features from a combination of figures.

### DETAILED DESCRIPTION

The present teachings describe a core-shell catalyst for a red-emission having a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle for the red-emission emits a red-emission upon a near-infrared (NIR) excitation. In some aspects, the UCNP shell further comprises gadolinium (Gd).

Some embodiments disclose approaches for integration of NVNDs with LiYF₄:Yb, Er UNCPs for enhancing optical manipulation and enabling efficient energy transfer, which improves existing quantum sensing technologies by offering a more efficient, cost-effective solution by use of cleaned NVNDs and opens new avenues for applications in biological imaging and quantum sensing due to upconversion capabilities of UNCPs. The disclosure significantly advances over currently known technologies by a novel approach that combines NVNDs with UNCPs in the disclosed core-shell nanoparticle wherein the NVND core is surrounded by a UNCP shell for enhanced laser cooling and quantum sensing. The disclosed core-shell combination leverages the unique energy transfer properties of UNCPs to improve the modulation of NV center emissions and offers a more efficient and versatile method for controlling optical properties of NV centers as compared to modulation of NV centers by other methods such as a nanodiamond coupled to a cylindrical silicon nanoantenna, thus broadening the potential application in quantum technologies. The core-shell nanoparticle can alternatively be considered as the core being encompassed, or encased, or encapsulated by the shell.

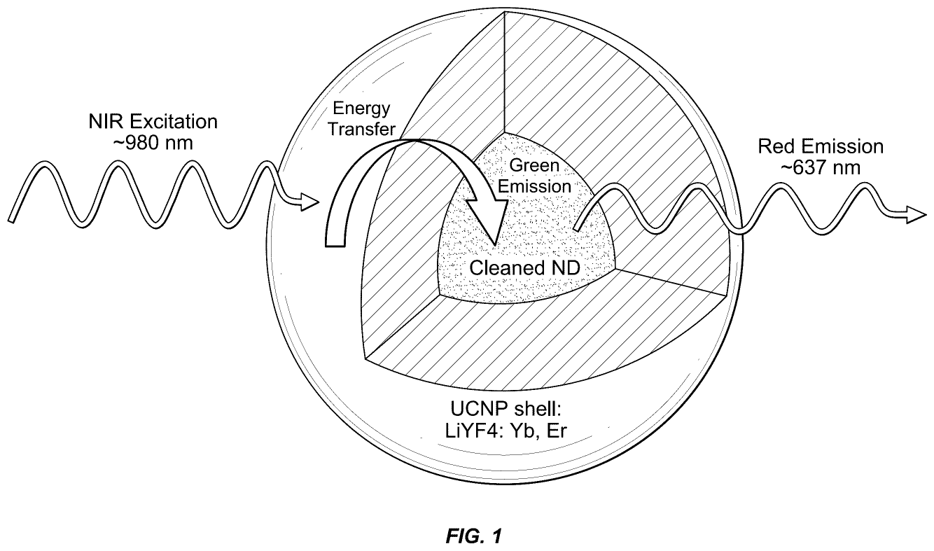

FIG. 1 is a schematic illustration of an NIR-excited core-shell nanoparticle comprising a cleaned nanodiamond (ND) core and an upconversion nanoparticle (UNCP) shell. FIG. 1 demonstrates the effect of integration of NVNDs and UNCPs on energy transfer processes leading to red emission from NV centers upon near-infrared (NIR) excitation of the core-shell nanoparticle. The core of the core-shell nanoparticle is a nanodiamond that has undergone an advanced cleaning process involving oxidation in molten potassium nitrate, which removes graphitic and non-diamond carbon impurities, thus enhancing its fluorescent properties, and the purity of the cleaned ND is greater than 99%. This cleaned ND core is enveloped by a UCNP shell made of lithium yttrium fluoride (LiYF₄) doped with lanthanide ions such as Ytterbium (Yb) and erbium (Er). Upon near-infrared (NIR) excitation at a wavelength of 980 nm, the UCNP shell absorbs the light because of Yb and, through a process of energy transfer, upconverts it to visible emissions (520-550 nm:green band due to Er). The energy from the UNCP is then transferred to the cleaned ND core, resulting in a red emission characteristic of the nitrogen-vacancy (NV) centers within the diamond. This red emission occurs at approximately 637 nm, which is within the spectral region of the NV centers' zero-phonon line (ZPL). The core-shell configuration of a cleaned ND core and a LiYF₄:Yb, Er UCNP shell harnesses the advanced luminescence properties of cleaned NDs combined with the energy upconversion capabilities of the UCNP shell, illustrating the novel approach to enhancing diamond fluorescence for potential applications in bio-labeling, quantum sensing, and integrated photonic devices.

In some aspects, the near-infrared (NIR) excitation is performed at about 805 nm for UNCP shell having neodymium (Nd) in the lanthanide ion combination.

Upon NIR excitation, the upconversion nanoparticle (UCNP) upconverts the near-infrared (NIR) excitation of the UCNP shell to a UCNP-emission such as a green-emission ranging from about 523 nm to about 555 nm, a blue-emission ranging from about 450 nm to about 475 nm, or a red-emission of about 650 nm. For example, a lanthanide ion combination of Yb and Er shows emission peaks of about 523 nm (green) and about 550 nm (green). For example, a lanthanide ion combination of Yb and Tm shows emission peaks of about 450 nm (blue) and about 475 nm (blue). For example, a lanthanide ion combination of Yb and Ho shows emission peaks of about 540 nm (green) and about 650 nm (red). Gadolinium in the UNCP shell acts as a host matrix. In some aspects, the UCNP shell further comprises gadolinium (Gd).

The performance of the disclosed core-shell nanoparticles is significantly dependent on the efficiency of energy transfer from the upconversion nanoparticles (UCNPs) to the nitrogen-vacancy (NV) centers within nanodiamonds. Quantum optics principles dictate that this energy transfer is important and can be quantified through Förster Resonance Energy Transfer (FRET) efficiency. The efficiency equation E=1/(1+r/R₀)⁶) illustrates this relationship, where ‘E’ denotes the energy transfer relationship, ‘r’ represents the distance between the UNCP donor and NV center acceptor, and ‘R₀’ is the Förster distance.

High FRET efficiency indicates that a greater proportion of energy from the UCNPs is transferred to the NV centers, enhancing the signal-to-noise ratio (SNR) of Optically Detected Magnetic Resonance (ODMR) measurements. The SNR is also influenced by the lifetime of the excited state (τ), inversely proportional to the square root of the lifetime, described as SNR 1/√τ.

An increase in SNR improves the ODMR contrast, which is crucial for the distinctiveness and accuracy of measurements. The ODMR contrast ‘C’ is defined by the equation C=ΔI/I, where ‘ΔI’ is the change in intensity due to microwave irradiation, and ‘I’ is the baseline intensity. An improved SNR means that the change in intensity ‘ΔI’ is more distinguishable from baseline ‘I’, thus enhancing the contrast and overall effectiveness of the disclosed core-shell nanoparticles having the NVND core and UNCP shell system in its various quantum sensing related applications.

Efficient Energy Transfer from the disclosed core-shell nanoparticle: FIG. 1 shows that the UCNP shell, composed of lithium yttrium fluoride (LiYF₄) doped with ytterbium (Yb) and erbium (Er), is engineered to absorb near-infrared (NIR) light and upconvert it to visible light. This light is then transferred to the NV centers in the diamond core, resulting in efficient red emission characteristic of the NV centers. This innovative approach enables the system to utilize NIR light effectively, making it suitable for applications requiring deep tissue penetration or minimal photodamage.

Enhanced Laser Cooling Capability of the disclosed core-shell nanoparticle: The disclosed core-shell structures of NVND core with LiYF₄:Yb, Er UNCP shell offers superior laser cooling efficiency. This is due to the enhanced energy transfer from UCNPs to NVNDs, enabling effective cooling in physiological media, which is a breakthrough in localized cooling applications.

Quantum Sensing Improvement: Beyond laser cooling, this novel configuration of the disclosed core-shell nanoparticle significantly improves quantum sensing capabilities. The precise energy modulation provided by the UCNP shell expands the operational range and sensitivity of NV centers, offering broad implications for quantum technology applications.

The size and shape of particles having the core-shell nanoparticles can be optimized to maximize total surface area of the particle and the number of active sites available for energy transfer per volume of particle used. In some examples, a diameter of the core-shell nanoparticle ranges from about 10 nm to about 200 nm, about 20 nm to about 100 nm, or about 30 nm to about 50 nm.

In some examples, the particles of a core-shell nanoparticle will include homogenous, nonporous particles.

The shape of the core-shell nanoparticles can be polyhedral, nanorod, nanowire, nanoplate or nanosheet. The polyhedral shape of the core-shell nanoparticles can be spherical, cubic, octahedral, cube, tetrahedral, dodecahedral, icosahedral, or cuboctahedral or other geometric shapes.

In some examples, the core-shell nanoparticle, with an NVND core and LiYF₄:Yb, Er, Tm UCNP shell, is subjected to near-infrared (NIR) excitation specifically at around 980 nm. In some examples, the near-infrared (NIR) excitation is about 980 nm for a UNCP comprising ytterbium (Yb) in the lanthanide ion combination. In some examples, the near-infrared (NIR) excitation is about 808 nm for a UNCP comprising neodymium (Nd) in the lanthanide ion combination.

In some examples, the LiYF₄:Yb, Er, Tm UCNP upconverts the about 980 nm near-infrared (NIR) excitation of the core-shell nanoparticle to green emission peaks, including approximately 523 nm and 550 nm (the strongest peak). In some examples, the LiYF₄:Yb, Er, Tm UCNP shell emits light at various wavelengths, including approximately 457 nm, 523 nm, 550 nm, 667 nm, and 792 nm following NIR excitation at about 980 nm.

In some examples, the cleaned nitrogen-vacancy nanodiamond (NVND) core converts a green emission from the UCNP shell to a red emission ranging from about 630 nm to about 645 nm. The ZPL of the NV center of the cleaned NVND core occurs at about 637 nm.

In some examples, the cleaned nitrogen-vacancy nanodiamond (NVND) comprises a nitrogen-vacancy (NV) center, and a distance (r) between the nitrogen-vacancy center and the UCNP ranges from about 2 nm to about 10 nm, from about 3 m to about 9 nm, from about 4 nm to about 8 nm, from about 5 nm to about 7 nm, from about 5 nm to about 6 nm, from about 6 nm to about 7 nm, about 2 nm, about 2.5 nm, about 3 nm, about 3.5 nm, about 4 nm, about 4.5 nm, about 5 nm, about 5.5 nm, about 6 nm, about 6.5 nm, about 7 nm, about 7.5 nm, about 8 nm, about 8.5 nm, about 9 nm, about 9.5 nm, or about 10 nm.

In the disclosed core-shell nanoparticles, performance of the disclosed core-shell nanoparticles depends on the efficiency of energy transfer from the upconversion nanoparticles (UCNPs) to the nitrogen-vacancy (NV) centers within nanodiamonds. The disclosed core-shell nanoparticles can have a Forster Resonance Energy Transfer (FRET) efficiency of about 30% to about 70%, about 40% to about 60%, or about 45% to about 55%.

In some examples, a diameter of the core-shell nanoparticle ranges from about 10 nm to about 200 nm, about 20 nm to about 100 nm, or about 30 nm to about 50 nm.

In some examples, a doping percentage of lanthanides in the upconversion nanoparticles can be about 0.1% to about 20% by weight. In some examples of the core-shell nanoparticle, the UCNP comprises a ratio of Y:Yb:Er ranging from about 70:25:5 to about 78:20:2 by weight.

The surface area of the disclosed core-shell nanoparticles is maximized for enhanced interaction with external agents, and the functionalization includes groups such as amino, thiol, carboxyl, hydroxyl, silane, or phosphate groups. In some examples of the disclosed core-shell nanoparticle, the cleaned nitrogen-vacancy nanodiamond (NVND) is conjugated with a group chosen from a carboxylic acid, a modified carboxylic acid, a hydroxyl, or a modified hydroxylic acid. In some examples of the disclosed core-shell nanoparticle, the UCNP is conjugated with a group chosen from amino group, a thiol group, a silane group, or a phosphate group. In some examples of the disclosed nanoparticle, the NVND is covalently conjugated with the UCNP.

In some examples, a film comprising the disclosed core-shell nanoparticles is provided. Such films can have uses in biomedical applications including drug delivery, tissue engineering and biosensing. Disclosed films can also have uses as coatings for protective eyewear, antifogging or architectural glass. A thin film of the disclosed core-shell nanoparticles may be applied to an active microelectronics device for near-junction (hot spot) cooling applications.

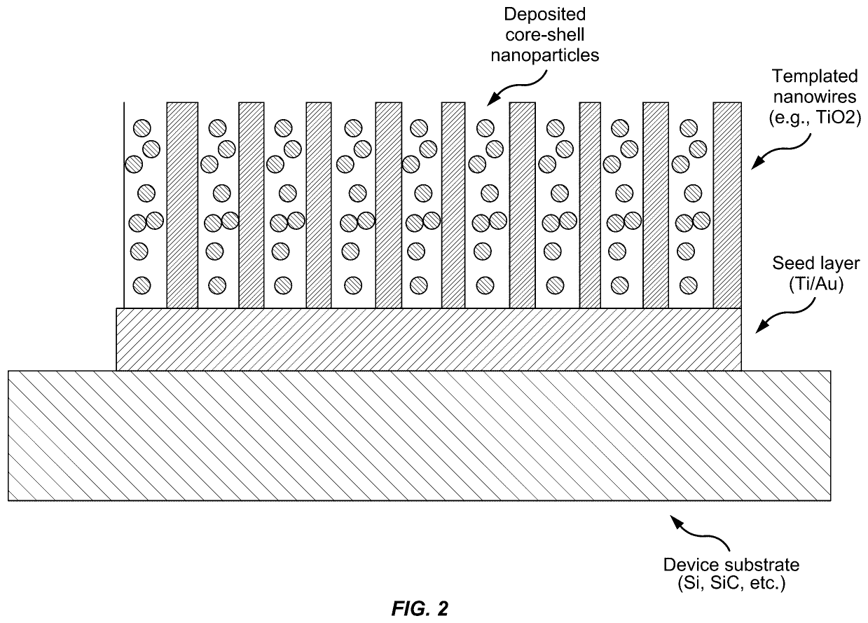

FIG. 2 provides an application having a film prepared with the disclosed core-shell nanoparticles. A thin film of the disclosed core-shell nanoparticles may be applied to an active microelectronics device for near-junction (hot spot) cooling applications. In FIG. 2 is a schematic view of a proposed application of disclosed core-shell nanoparticle film in a device illustrating a device substrate (Si, SiC, etc.), seed layer (Ti/Au), templated nanowires (eg. TiO₂) and film of deposited core-shell nanoparticles is provided.

In some examples, a suspension comprising the disclosed core-shell nanoparticle is provided. A suspension of nanoparticles refers to a colloidal system in which nanoparticles are dispersed or suspended in a liquid medium, such as water, oil, or a solvent. Nanoparticle suspensions having the disclosed core-shell nanoparticles can be used as carriers for delivering drugs, genes, or therapeutic agents to specific target sites in the body. Surface-modified nanoparticles can enhance drug solubility, bioavailability, and targeting efficiency. Nanoparticle suspensions having the disclosed core-shell nanoparticles can serve as contrast agents in medical imaging techniques such as magnetic resonance imaging (MRI), computed tomography (CT), and fluorescence imaging. Nanoparticle suspensions having the disclosed core-shell nanoparticles can be utilized in various therapeutic applications, including cancer therapy, antimicrobial treatment, and regenerative medicine.

In some examples, a bio-labeled molecule for detection of red-emission comprising a biomolecule labeled with the disclosed core-shell nanoparticle is provided. The disclosed bio-labeled molecules can be used for detection of red-emission from a bio-imaging device. The bioimaging device can be a non-invasive imaging device, a magnetic resonance imaging device, or a computer assisted tomography device.

In some examples, the bio-labeled molecule for detection of red-emission comprising a biomolecule labeled with the disclosed core-shell nanoparticle can be detected at a biological tissue depth ranging from about 0.5 millimeters to about 1.5 centimeters, about 1 millimeter to about 1 centimeter, or about 2 millimeters to about 5 millimeters.

In some aspects, a bio-labeled molecule for quantum laser-cooling comprising a biomolecule labeled with the disclosed core-shell nanoparticle is disclosed. The bio-labeled molecule for quantum laser-cooling enables laser cooling to temperatures ranging from about 5° C. to about 25° C., about 10° C. to about 20° C., or about 12° C. to about 18° C. in physiological media.

In some aspects, a bio-labeled molecule labeled with the disclosed core-shell nanoparticles for use in a treatment method for treating a subject in need thereof is provided. In some aspects, a quantum-sensing molecule comprising a core-shell nanoparticle is disclosed, wherein the core-shell nanoparticle comprises a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy (NV) nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation. In some aspects, the UCNP shell further comprises gadolinium (Gd). In the quantum-sensing molecule the cleaned nitrogen-vacancy nanodiamond (NVND) comprises a nitrogen-vacancy center, and wherein a distance (r) between the nitrogen-vacancy center and the UCNP is about 2 nm to about 10 nm.

In some aspects, the quantum-sensing molecule is applicable for measurement of a quantum sensing parameter in a method chosen from precision metrology, navigation, magnetic field sensing, or quantum information processing. In some aspects, the quantum-sensing molecule is useful for application a quantum-sensor device, an advanced quantum sensor device, a quantum-cooler device, a spin-sensing device, a magnetic sensing device, or a bio-imaging device. The advanced quantum sensor device can be chosen from quantum-enhanced imaging device, a quantum-enhanced spectroscopy system, or a quantum-sensor integrated into a quantum computing platform. The quantum-sensing application can be chosen from precision metrology, navigation, magnetic field sensing, or quantum information processing.

Some aspects relate to a quantum-cooler molecule comprising a core-shell nanoparticle, wherein the core-shell nanoparticle a cleaned nitrogen-vacancy nanodiamond (NVND) core surrounded by an upconversion nanoparticle (UCNP) shell, wherein the cleaned nitrogen-vacancy nanodiamond (NVND) core comprises a cleaned nitrogen-vacancy nanodiamond, wherein the UCNP shell comprises an upconversion nanoparticle (UCNP), wherein the UCNP comprises lithium yttrium fluoride (LiYF₄) doped with a lanthanide ion combination, wherein the lanthanide ion combination comprises a M1 ion and a M2 ion, wherein M1 is ytterbium (Yb) or neodymium (Nd) and M2 is erbium (Er), thulium (Tm) or holmium (Ho), and wherein the core-shell nanoparticle emits a red-emission upon a near-infrared (NIR) excitation. In some aspects, the UCNP shell further comprises gadolinium (Gd).

In some aspects, the quantum-cooler molecule can lower temperature of a system to a range of about 1 kelvin to about 100 kelvins.

Some aspects provide a quantum-cooler molecule for a laser-cooling application chosen from observing quantum phenomena (such as Bose-Einstein condensation or superfluidity), for an application in quantum information processing, for an application in quantum simulation, or for an application in precision metrology.

Some aspects provide a device for detection of the disclosed core-shell nanoparticles. Such devices can be biomedical imaging device for detecting and imaging biological tissues, quantum computing device for quantum information processing, precision metrology device for high-resolution measurements, magnetic field sensor for detecting and mapping magnetic fields, single-photon detector for quantum communication and cryptography, laser cooling system for achieving low temperatures in quantum systems, or an atom interferometry device for precision measurements and fundamental physics experiments.

### Examples

Various aspects of the present disclosure are further illustrated with respect to the following examples. It is to be understood that these examples are provided to illustrate specific examples of the present disclosure and should not be construed as limiting the scope of the present disclosure in or to any particular aspect.

The present disclosure is not limited to the above aspects or examples but can be implemented by any of various other aspects or examples within the scope of the disclosure.

Further, the disclosure comprises additional notes and examples as detailed below.
Clause 1. A core-shell nanoparticle comprising a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.
Clause 2. The core-shell nanoparticle of Clause 1, wherein the UCNP shell further comprises gadolinium (Gd).
Clause 3. The core-shell nanoparticle of Clause 1, wherein the near-infrared (NIR) excitation is about 980 nm for ytterbium (Yb).
Clause 4. The core-shell nanoparticle of Clause 1, wherein the near-infrared (NIR) excitation is about 808 nm for neodymium (Nd).
Clause 5. The core-shell nanoparticle of Clause 1, wherein the LiYF₄:Yb, Er, Tm UCNP is excited by near-infrared (NIR) light specifically at around 980 nm.
Clause 6. The core-shell nanoparticle of Clause 1, wherein the near-infrared (NIR) excitation is about 980 nm.
Clause 7. The core-shell nanoparticle of Clause 1, wherein the upconversion nanoparticle (UCNP) upconverts the near-infrared (NIR) excitation of the core-shell nanoparticle to a green-emission ranging from about 523 nm to about 555 nm, a blue-emission ranging from about 450 nm to about 475 nm, or a red-emission of about 650 nm.
Clause 8. The core-shell nanoparticle of Clause 1, wherein the upconversion nanoparticle (UCNP) upconverts the near-infrared (NIR) excitation of the core-shell nanoparticle to a green-emission ranging from about 523 nm to about 555 nm.
Clause 9. The core-shell nanoparticle of Clause 1, wherein the upconversion nanoparticle (UCNP) upconverts the near-infrared excitation of the UNCP shell to green-emission of about 520 nm.
Clause 10. The core-shell nanoparticle of Clause 1, wherein the upconversion nanoparticle (UCNP) upconverts the near-infrared excitation of the UNCP shell to green-emission of about 523 nm.
Clause 11. The core-shell nanoparticle of Clause 1, wherein the cleaned nitrogen-vacancy nanodiamond (NDNV) core converts a green-emission from the UNCP shell to a red-emission ranging from about 630 nm to about 645 nm.
Clause 12. The core-shell nanoparticle of Clause 1, wherein the UCNP comprises a ratio of Y:Yb:Er ranging from about 70:25:5 to about 78:20:2 by weight.
Clause 13. The core-shell nanoparticle of Clause 1, wherein the cleaned nitrogen-vacancy nanodiamond (NDNV) core converts a green-emission from the UCNP shell to a red-emission of about 637 nm.
Clause 14. The core-shell nanoparticle of Clause 1, wherein the cleaned nitrogen-vacancy nanodiamond (NVND) comprises a nitrogen-vacancy center in the cleaned nitrogen-vacancy nanodiamond, and wherein the core-shell nanoparticle emits spectra within zero phonon line (ZPL) of about 637 nm of the nitrogen-vacancy center upon the near-infrared (NIR) excitation of the core-shell nanoparticle.
Clause 15. The core-shell nanoparticle of Clause 1, wherein the cleaned nitrogen-vacancy nanodiamond (NVND) comprises a nitrogen-vacancy (NV) center and wherein a distance (r) between the nitrogen-vacancy center and the UCNP is about 2 nm to about 10 nm.
Clause 16. The core-shell nanoparticle of Clause 1 comprising a Forster Resonance Energy Transfer (FRET) efficiency of 30% to about 70%, about 40% to about 60%, or about 45% to about 55%.
Clause 17. The core-shell nanoparticle of Clause 1 comprising a diameter of the core-shell nanoparticle ranging from about nm 10 to about 200 nm, about 20 nm to about 100 nm, or about 30 nm to about 50 nm.
Clause 18. The core-shell nanoparticle of Clause 1, wherein the UCNP comprises a ratio of Y:Yb:Er ranging about 70:25:5 to about 78:20:2.
Clause 19. The core-shell nanoparticle of Clause 1, wherein the cleaned nitrogen-vacancy nanodiamond (NVND) is conjugated with a group chosen from a carboxylic acid, a modified carboxylic acid, a hydroxyl, or a modified hydroxylic acid.
Clause 20. The core-shell nanoparticle of Clause 1, wherein the UCNP is conjugated with a group chosen from with a group chosen from a carboxylic acid, a modified carboxylic acid, a hydroxyl, or a modified hydroxylic acid.
Clause 21. The core-shell nanoparticle of Clause 1, wherein the NVND is covalently conjugated with a UCNP.
Clause 22. The core-shell nanoparticle of Clause 1 having a shape selected from the group consisting of polyhedral, nanorod, nanowire, nanoplate or nano sheet.
Clause 23. The core-shell nanoparticle of clause shape, wherein the polyhedral shape is selected from the group consisting of spherical, cubic, octahedral, cube, tetrahedral, dodecahedral, icosahedral, cuboctahedral or other geometric shapes.
Clause 24. A film comprising a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.
Clause 25. A suspension comprising a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.
Clause 26. A bio-labeled molecule for detection of red-emission comprising a biomolecule labeled with a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.
Clause 27. The bio-labeled molecule for detection of red-emission of Clause 26 for detection of red-emission from a bio-imaging device.
Clause 28. The bio-labeled molecule for detection of red-emission of Clause 27, wherein the bio-imaging device is chosen from a non-invasive imaging device, a magnetic resonance imaging device, or computer assisted tomography device.
Clause 29. The bio-labeled molecule of Clause 26, wherein the bio-labeled molecule can be detected at a biological tissue depth ranging from about 0.5 millimeters to about 1.5 centimeters, about 1 millimeter to about 1 centimeter, or about 2 millimeters to about 5 millimeters.
Clause 30. A bio-labeled molecule for quantum laser-cooling comprising a biomolecule labeled with core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy (NV) nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation, and
wherein the bio-labeled molecule enables laser cooling ranging from about 5° C. to about 25° C., about 10° C. to about 20° C., or about 12° C. to about 18° C. in physiological media.
Clause 31. The bio-labeled molecule of Clause 30 for use in a treatment method for treating a subject in need thereof.
Clause 32. A quantum-sensing molecule comprising a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy (NV) nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.
Clause 33. The quantum-sensor molecule of Clause 32 for a quantum-sensing application chosen from precision metrology, navigation, magnetic field sensing, spin sensing, or quantum information processing.
Clause 34. The quantum-sensing molecule of Clause 33, wherein the cleaned nitrogen-vacancy nanodiamond (NVND) comprises a nitrogen-vacancy center wherein a distance (r) between the nitrogen-vacancy center and the UCNP is about 2 nm to about 10 nm.
Clause 35. A quantum-cooler molecule comprising a core-shell nanoparticle,
wherein the core-shell nanoparticle a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.
Clause 36. The quantum-cooler molecule of Clause 35 for a laser-cooling application chosen from observing quantum phenomena (such as Bose-Einstein condensation or superfluidity), for an application in quantum information processing, for an application in quantum simulation, or for an application in precision metrology.
Clause 37. The quantum-cooler molecule of Clause e 36 for lowering temperature to a range of about 1 Kelvin to about 100 Kelvin of a quantum system.
Clause 38. A device for detection of a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.
Clause 39. A biomedical imaging device for detecting a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), is ytterbium (Yb) and erbium (Er), and
wherein the core-shell nanoparticle emits a red-emission upon a near-infrared (NIR) excitation.
Clause 40. A biomedical imaging device for imaging biological tissues by detecting a core-shell nanoparticle, wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), is ytterbium (Yb) and erbium (Er), and
wherein the core-shell nanoparticle emits a red-emission upon a near-infrared (NIR) excitation.
Clause 41. A quantum computing device for quantum information processing by detecting a core-shell nanoparticle, wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), is ytterbium (Yb) and erbium (Er), and
wherein the core-shell nanoparticle emits a red-emission upon a near-infrared (NIR) excitation.
Clause 42. A precision meterology device for high-resolution measurements by detecting a core-shell nanoparticle, wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), is ytterbium (Yb) and erbium (Er), and
wherein the core-shell nanoparticle emits a red-emission upon a near-infrared (NIR) excitation.
Clause 43. A magnetic field sensor for detecting and mapping magnetic fields by detecting a core-shell nanoparticle, wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), is ytterbium (Yb) and erbium (Er), and
wherein the core-shell nanoparticle emits a red-emission upon a near-infrared (NIR) excitation.
Clause 45. A single photon detector for quantum communication and cryptography by detecting a core-shell nanoparticle, wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), is ytterbium (Yb) and erbium (Er), and
wherein the core-shell nanoparticle emits a red-emission upon a near-infrared (NIR) excitation.
Clause 46. A laser cooling system for achieving low temperatures in quantum systems by detecting a core-shell nanoparticle, wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), is ytterbium (Yb) and erbium (Er), and
wherein the core-shell nanoparticle emits a red-emission upon a near-infrared (NIR) excitation.
Clause 47. An atom interferometry device for precision measurements and fundamental physics experiments by detecting a core-shell nanoparticle, wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), is ytterbium (Yb) and erbium (Er), and
wherein the core-shell nanoparticle emits a red-emission upon a near-infrared (NIR) excitation.

The preceding description is merely illustrative in nature and is in no way intended to limit the disclosure, its application, or uses. As used herein, the phrase at least one of A, B, and C should be construed to mean a logical (A or B or C), using a non-exclusive logical “or.” It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure and are not intended to limit the disclosure of the technology or any aspect thereof. The recitation of multiple examples having stated features is not intended to exclude other embodiments having additional features, or other examples incorporating different combinations of the stated features.

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that an example can or may comprise certain elements or features does not exclude other examples of the present technology that do not contain those elements or features.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one aspect, or various aspects means that a particular feature, structure, or characteristic described in connection with an embodiment or particular system is included in at least one embodiment or aspect. The appearances of the phrase “in one aspect” (or variations thereof) are not necessarily referring to the same aspect or embodiment. It should be also understood that the various method steps discussed herein do not have to be carried out in the same order as depicted, and not each method step is required in each aspect or embodiment.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations should not be regarded as a departure from the disclosure, and all such modifications are intended to be included within the scope of the disclosure.

What is claimed is:

1. A core-shell nanoparticle comprising a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.

2. The core-shell nanoparticle of claim 1, wherein the UCNP shell further comprises gadolinium (Gd).

3. The core-shell nanoparticle of claim 1, wherein the near-infrared (NIR) excitation is about 980 nm for ytterbium (Yb).

4. The core-shell nanoparticle of claim 1, wherein the near-infrared (NIR) excitation is about 808 nm for neodymium (Nd).

5. The core-shell nanoparticle of claim 1, wherein the upconversion nanoparticle (UCNP) upconverts the near-infrared (NIR) excitation of the UCNP shell to a UCNP-emission of a green-emission ranging from about 523 nm to about 555 nm, or a blue-emission ranging from about 450 nm to about 475 nm, or a red-emission of about 650 nm.

6. The core-shell nanoparticle of claim 1, wherein the cleaned nitrogen-vacancy nanodiamond (NDNV) core converts the UNCP-emission to a red-emission ranging from about 630 nm to about 645 nm.

7. The core-shell nanoparticle of claim 1, wherein the UCNP comprises a ratio of Y:Yb:Er ranging from about 70:25:5 to about 78:20:2 by weight.

8. The core-shell nanoparticle of claim 1, wherein the cleaned nitrogen-vacancy nanodiamond (NVND) comprises a nitrogen-vacancy (NV) center, and a distance (r) between the NV center and the UCNP ranges from about 2 nm to about 10 nm.

9. The core-shell nanoparticle of claim 1 comprising a Forster Resonance Energy Transfer (FRET) efficiency ranges from about 30% to about 70%.

10. The core-shell nanoparticle of claim 1 comprising a diameter of the core-shell nanoparticle ranging from about 10 nm to about 200 nm.

11. A film comprising a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.

12. A suspension comprising a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.

13. A bio-labeled molecule for detection of red-emission comprising a biomolecule labeled with a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.

14. The bio-labeled molecule for detection of red-emission of claim 13 for detection of red-emission from a bio-imaging device.

15. The bio-labeled molecule for detection of red-emission of claim 11, wherein the bio-imaging device is chosen from a non-invasive imaging device, a magnetic resonance imaging device, or computer assisted tomography device.

16. The bio-labeled molecule of claim 14, wherein the bio-labeled molecule can be detected at a biological tissue depth ranging from about 0.5 millimeters to about 1.5 centimeters.

17. A bio-labeled molecule for quantum laser-cooling comprising a biomolecule labeled with core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy (NV) nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation, and
wherein the bio-labeled molecule enables laser cooling ranging from about 5° C. to 25° C. in physiological media.

18. A quantum-sensing molecule comprising a core-shell nanoparticle,
wherein the core-shell nanoparticle comprises a cleaned nitrogen-vacancy nanodiamond (NVND) core surrounded by an upconversion nanoparticle (UCNP) shell,
wherein the cleaned nitrogen-vacancy nanodiamond (NVND) core comprises a cleaned nitrogen-vacancy (NV) nanodiamond,
wherein the UCNP shell comprises an upconversion nanoparticle (UCNP),
wherein the UCNP comprises lithium yttrium fluoride (LiYF₄) doped with a lanthanide ion combination,
wherein the lanthanide ion combination comprises a M1 ion and a M2 ion, wherein M1 is ytterbium (Yb) or neodymium (Nd) and M2 is erbium (Er), thulium (Tm) or holmium (Ho), and
wherein the core-shell nanoparticle emits a red-emission upon a near-infrared (NIR) excitation.

19. The quantum-sensing molecule of claim 16 for measurement of a quantum sensing parameter in a method chosen from precision metrology, navigation, magnetic field sensing, or quantum information processing.

20. The quantum-sensing molecule of claim 18, wherein the cleaned nitrogen-vacancy nanodiamond (NVND) comprises a nitrogen-vacancy (NV) center, and wherein a distance (r) between the NV center and the UCNP ranges from about 2 nm to about 10 nm.
